(19) United States
(12) Patent Application Publication
(10) Pub. No.: US 2024/0277433 A1
(43) Pub. Date: Aug. 22, 2024

(54) METHOD FOR CONDITIONING A SURGICAL INSTRUMENT OF A ROBOTIC SURGERY SYSTEM, WITH CYCLES OF PRE-ELONGATION OF MOVEMENT TRANSMISSION TENDONS, AND RELATED ROBOTIC SYSTEM

(71) Applicant: MEDICAL MICROINSTRUMENTS, INC., Wilmington, DE (US)

(72) Inventors: Matteo TANZINI, Pisa (IT); Massimiliano SIMI, Pisa (IT); Giuseppe Maria PRISCO, Pisa (IT); Michael John PROCTOR, Pisa (IT)

(21) Appl. No.: 18/570,404

(22) PCT Filed: Jun. 16, 2022
(86) PCT No.: PCT/IB2022/055576

§ 371 Date: Dec. 14, 2023

(30) Foreign Application Priority Data
Jun. 17, 2021 (IT) 102021000015896

(51) Int. Cl. A61B 34/37; A61B 34/00

(52) U.S. Cl. CPC A61B 34/37; A61B 34/71; A61B 2034/715

(57) ABSTRACT

A method conditions a surgical instrument of a robotic surgery system prior to use. An articulated end-effector has degree(s) of freedom, and a tendon, operatively connects with a motorized actuator of the robotic surgery system. The tendon mounts to the surgical instrument to connect to both a respective motorized actuator, among transmission elements, and degree(s) of freedom of the end-effector. The degree(s) of freedom are activated by respective motorized actuator action by the connected tendon. The method includes locking a degree(s) of freedom of the end-effector; tensile-stressing the respective tendon, operatively connecting the locked degree of freedom, by applying force, according to a time cycle, to the tendon. The time cycle includes a low-load period, in which a low conditioning force Flow is applied, which results in a low tensile load on the tendon; and a high-load period, in which force is applied, which results in high tensile load.

### TECHNOLOGICAL BACKGROUND OF THE INVENTION

### Field of Application

The present invention relates to a method for conditioning of a surgical instrument of a robotic surgery system, with cycles of pre-elongation of movement transmission tendons.

Therefore, the present description relates, more generally, to the technical field of operational control of robotic systems for teleoperated surgery.

### Description of the Prior Art

In a teleoperated robotic surgery system the actuation of one or more degrees of freedom of a slave surgical instrument is generally enslaved to one or more master control devices configured to receive a command imparted by the surgeon. Such a master-slave control architecture typically comprises a control unit which can be housed in the robotic surgery robot.

Known articulated surgical instruments for robotic surgery systems include actuation tendons or cables for transmitting motion from the actuators, operatively connected to proximal interface (or “backend”) of the surgical instrument, distally to the tips of the surgical instrument intended to operate on a patient anatomy and/or to handle a surgical needle, as for example shown in documents WO-2017-064303 and WO-2018-189729 in the name of the same Applicant. Such documents disclose solutions in which a pair of antagonistic tendons is configured to implement the same degree of freedom as the surgical instrument. For example, a rotational joint of the surgical instrument (degree of freedom of pitch and degree of freedom of yaw) is controlled by applying tensile force applied by the pair of the aforesaid antagonistic tendons.

Further known are surgical instruments in which the same pair of tendons is capable of simultaneously actuating more than one degree of freedom, such as shown in WO-2010-009221 in which only two pairs of tendons are configured to control three degrees of freedom of the surgical instrument.

Typically, tendons for robotic surgery are made in the form of metal cords (or strands) and are wound around pulleys mounted along the surgical instrument. Each tendon can be mounted on the already elastically preloaded instrument, i.e., pre-conditioned prior to assembly on the instrument, so that each tendon is always in a tensile state in order to provide a rapid actuation response of the degree of freedom of the surgical instrument when activated by the actuators and, consequently, to provide good control over the degree of freedom of the surgical instrument.

For example, document US-2019-0191967 shows a pre-elongation solution which includes pulling both antagonistic tendons at the same speed at the same time, until the pulling motors begin to experience a significant increase in the torque generated by a minimum further movement, to identify the torque of the motor which determines the end of loosening transition (“no slack”) in the tendons.

In general terms, all the cords are subject to elongation when subjected to loads. New cords of the braided type typically have a high elongation of plastic-elastic nature when under load due at least in part to the unraveling of the fibers forming the cord.

For this reason, before assembly on the surgical instrument, it is common practice to subject the new tendons to a high initial load in order to remove the residual plasticity of the drawing and braiding process or of the material itself.

In general, the cords typically have three elongation elements:
(1) elastic elongation deformation, which is recovered when the tensile load stops;
(2) recoverable deformation, i.e., a relatively small deformation which is gradually recovered over a certain period of time and is often a function of the nature of the intertwinement, and can take a period of time between a few hours and a few days when not subjected to any load;
(3) non-recoverable permanent elongation deformation.

The permanent elongation deformation, as described above, can be achieved by a cord breaking-in procedure, performed prior to assembly on the instrument, which can comprise loading and unloading cycles and involve a plastic elongation deformation of the fibers themselves.

Viscous creep deformation under tensile load is a time-dependent effect which affects some types of intertwined cords when subject to fatigue and can be recoverable or non-recoverable typically depending on the intensity of the applied load.

Generally, the fatigue behavior of polymeric fibers differs from the fatigue behavior of metallic fibers in that the polymeric fibers are not subject to crack propagation breakage, as instead are metallic fibers, although cyclic stresses may lead to other forms of breakage.

WO-2017-064306 in the name of the same Applicant shows a solution of an extremely miniaturized surgical instrument for robotic surgery, which uses tendons adapted to support high radii of curvature and at the same time adapted to slide on the surfaces of the rigid elements, commonly called “links”, which form the articulated tip of the surgical instrument. In order to allow for such sliding of the tendons, the tendons-link sliding friction coefficient must be kept as low as possible, and the above-mentioned document teaches to use tendons formed by polymeric fibers (rather than using steel tendons).

Although advantageous from many points of view, and indeed as a consequence of the fact that an extreme miniaturization of the surgical instrument is obtained by virtue of the use of the aforesaid tendons formed by polymeric fibers, in the context of this solution it becomes even more important to avoid the occurrence of an elongation or a shortening (contraction) of the tendons under operating conditions of the surgical instrument, because with the same variation in length, as the size decreases, the uncontrollability effects of the miniaturized surgical instrument would be accentuated.

Metallic tendons have a modest recoverable elongation and the aforementioned preloading processes performed before assembly on the surgical instrument are typically sufficient to completely remove the residual plasticity, while the preload to which they are subject when assembled maintains an immediate reactivity in use.

For example, US-2018-0228563 shows a solution for eliminating the factory elastic elongation (“stretch”) of a tendon with a core and a plurality of fibers wound around the core. This solution includes subjecting each tendon to loading cycles so as to cause a relative sliding between the tendon fibers and between the tendon fibers and core, thus eliminating the empty spaces between the fibers and the core formed during the manufacture of the tendon.

Otherwise, the tendons in polymeric materials have high elongations due to the contributions described above; moreover, the preloading processes, if carried out before assembly, do not prevent the tendon from quickly recovering a large fraction of the recoverable elongation as soon as the tendons are subject to low tensile loads. If on the one hand the forecasting of any high assembly preloads prevents the recovery of the deformation, on the other hand it aggravates the creep process of the polymeric tendon even when not in use, forcing the tendon to stretch almost indefinitely and weaken, and therefore is not a viable strategy.

For example, braided cords formed by high molecular weight polyethylene fibers (HMWPE, UHMWPE) are usually subject to non-recoverable deformation, while braided cords of aramid, polyesters, liquid crystal polymers (LCP), PBO (Zylon®), nylon are less affected by this feature.

For example, WO-2017-064301 in the name of the same Applicant, shows a polymeric tendon of a surgical instrument capable of being subjected to pre-elongation cycles with a load at least equal to half the tensile breaking load of the tendon itself.

In the case of surgical instruments, the variation in the length of the tendons attributable to the tendon elongation phenomenon described above, as well as the recovery of the elongation, is highly undesirable, in particular when under operating conditions, because it would necessarily impose objective complications in the control in order to maintain an adequate level of precision and accuracy of the surgical instrument itself.

Therefore, the need is felt to avoid or at least minimize the lengthening/elongation of the actuation tendon of one or more degrees of freedom of the surgical instrument during use or over time, as well as to avoid, or at least minimize, the lost motion deriving from an undesirable lengthening/elongation of the tendon in operating conditions, such as during a teleoperation, without for this reason imposing an increase in the dimensions of the surgical instrument, particularly of the distal articulated portion thereof.

Meanwhile, the need is felt to provide a solution which, although simple, is capable of ensuring a high level of controllability of the surgical instrument, and is thus reliable when in operating conditions such as during a teleoperation, and meanwhile does not hinder a boosted miniaturization of the surgical instrument, especially in the distal articulated portion thereof.

### SUMMARY OF THE INVENTION

It is the object of the present invention to provide a conditioning method of a surgical instrument of a robotic surgery system, which allows to at least partially overcome the drawbacks claimed above with reference to the background art, and to respond to the aforementioned needs particularly felt in the technical field considered. Such an object is achieved by a method according to claim 1.

Further embodiments of such a method are defined in claims 2-16.

It is a further object of the present invention to provide a robotic surgery system capable of performing and/or adapted to be controlled by the aforesaid conditioning method of a surgical instrument. Such an object is achieved by a method according to claim 17.

Further embodiments of such a system are defined by claims 18-23.

### BRIEF DESCRIPTION OF THE DRAWINGS

Further features and advantages of the method according to the invention will become apparent from the following description of preferred exemplary embodiments, given by way of non-limiting indication, with reference to the accompanying drawings, in which:

FIG. 1 shows in axonometric view a robotic system for teleoperated surgery, according to an embodiment;

FIG. 2 shows in axonometric view a portion of the robotic system for teleoperated surgery of FIG. 1;

FIG. 3 shows in axonometric view a distal portion of a robotic manipulator, according to an embodiment;

FIG. 4 shows in axonometric view a surgical instrument, according to an embodiment, in which tendons are schematically diagrammatically shown in a dashed line;

FIG. 5 diagrammatically shows in plan and partially sectioned view for clarity the actuation of a degree of freedom of an articulated end-effector device (also called end-effector) of a surgical instrument, according to a possible operating mode;

FIG. 6 is a diagram which takes the example shown in FIG. 5 showing a step of a conditioning method, according to a possible operating mode;

FIG. 7 diagrammatically shows the actuation of a degree of freedom of an articulated end-effector of a surgical instrument, according to a possible operating mode;

FIG. 8 is a graph showing a conditioning force as a function of time, according to an operating mode;

FIG. 8b is a graph showing a position of a motorized actuator as a function of time, according to an operating mode;

FIG. 9 is a diagrammatic sectional view of a portion of a surgical instrument and a portion of a robotic manipulator showing the actuation of a degree of freedom of a surgical instrument, according to a possible operating mode;

FIG. 10 is a flow diagram showing steps of a conditioning method, according to a possible operating mode;

FIG. 11 is a flow diagram showing steps of a conditioning method, according to another possible operating mode;

FIG. 12 is a partially sectioned (for the sake of clarity) axonometric view showing an articulated end-effector of a surgical instrument, according to an embodiment;

FIG. 13 is a graph showing an application time cycle of a conditioning force as a function of time, according to an operating mode;

FIG. 14 comprises two graphs showing application time cycles of a conditioning force as a function of time, to two different tendons, according to an operating mode;

FIG. 15 comprises three graphs showing application time cycles of a conditioning force as a function of time, to three different pairs of antagonistic tendons, according to an operating mode;

FIG. 16 comprises two graphs (a) and (b) showing application time cycles of a conditioning force as a function of time, to two different pairs of antagonistic tendons, according to an operating mode;

FIG. 17 is a further graph showing application time cycles of a conditioning force as a function of time, to two different pairs of antagonistic tendons, according to an operating mode;

FIG. 18 is a graph which diagrammatically shows the trend over time of the force applied on a tendon, according to a possible operating mode;

FIG. 19 is a block diagram showing some possible steps of a method, according to an operating mode;

FIG. 20 is a graph showing an application time cycle of a conditioning force as a function of time, according to an operating mode.

### DETAILED DESCRIPTION

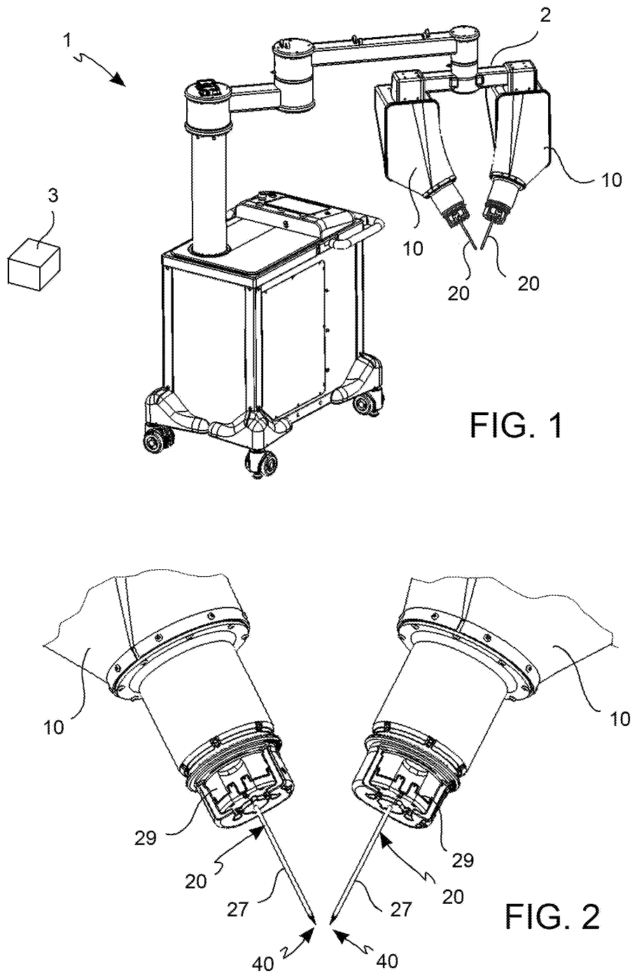

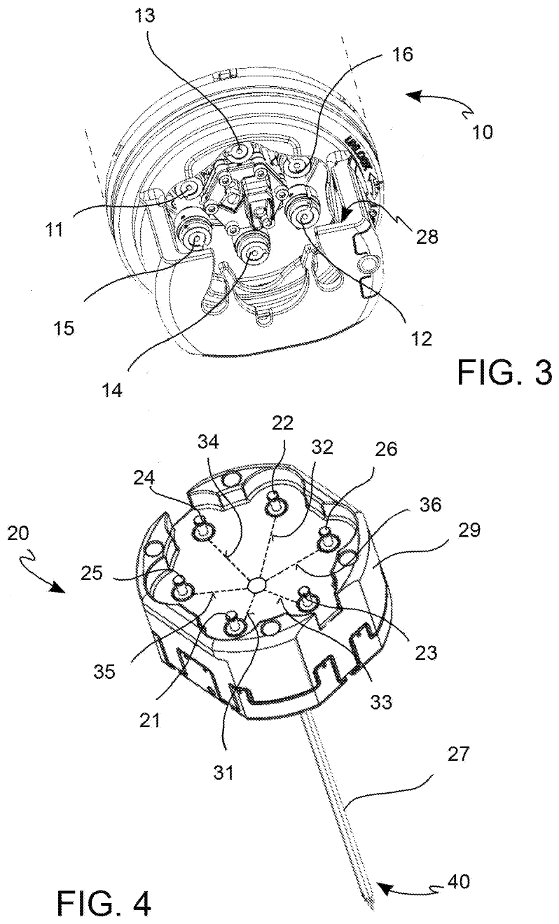

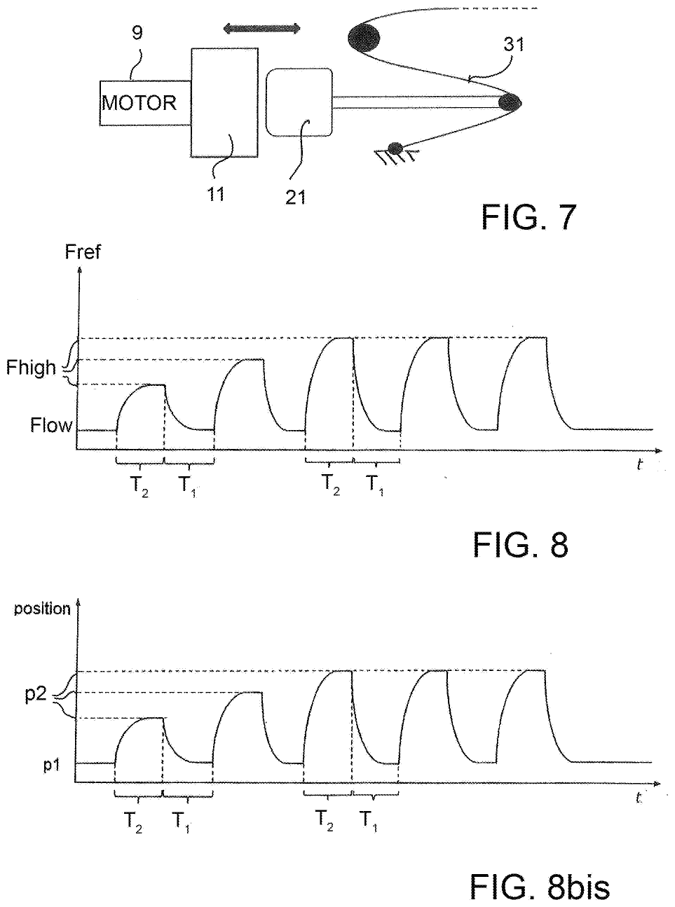

With reference to FIGS. 1-20, a method for conditioning a surgical instrument 20 of a robotic surgery system 1 is described.

The method can advantageously be performed on several situations before using the instrument, for example before packaging prior to a delivery of the instrument, or before being used for teleoperation. Preferably, the method is performed after mounting the surgical instrument to the robotic surgery system and before it is put into operation for a teleoperation.

The method is applied to a surgical instrument 20 comprising an articulated end-effector 40 having at least one degree of freedom (P, Y, G), and further comprises at least one tendon 31, 32, 33, 34, 35, 36, operatively connectable to a respective at least one motorized actuator 11, 12, 13, 14, 15, 16 of the robotic surgery system 1.

The at least one tendon 31, 32, 33, 34, 35, 36 is mounted to the surgical instrument 20 so as to be operatively connectable to both a respective motorized actuator, among the aforesaid at least one transmission element, and to at least one degree of freedom among the aforesaid at least one degree of freedom P, Y, G of the articulated end-effector 40.

The aforesaid at least one degree of freedom P, Y, G operatively connectable to the at least one tendon is adapted to be mechanically activated by an action of the at least one respective motorized actuator 11, 12, 13, 14, 15, 16 by means of the aforesaid at least one tendon 31, 32, 33, 34, 35, 36 which is operatively connectable thereto.

The method comprises the following steps:
(i) locking at least one degree of freedom of the aforesaid at least one degree of freedom P, Y, G of the end-effector 40;
(ii) tensile-stressing the respective at least one tendon, operatively connected to the aforesaid at least one locked degree of freedom, by applying a conditioning force (Fref), according to at least one time cycle, to the respective at least one tendon to be stressed under tensile load.

The aforesaid at least one time cycle comprises:
at least one low-load period, in which a low conditioning force Flow is applied to the aforesaid respective tendon, which results in a respective low tensile load on the respective tendon;
at least one high-load period, in which a high conditioning force Fhigh is applied to the aforesaid respective tendon, which results in a respective high tensile load on the respective tendon.

The method can be performed for example before using the surgical instrument.

According to a preferred implementation option, the method is applied to a surgical instrument 20 further comprising at least one transmission element 21, 22, 23, 24, 25, 26, which is operatively connectable to a respective at least one motorized actuator 11, 12, 13, 14, 15, 16 of the robotic surgery system 1. Thus, the transmission system of the surgical instrument 20 comprises the aforesaid at least one tendon, deformable under tensile load, and also the aforesaid at least one transmission element which interfaces with the aforesaid at least one respective motorized actuator of the robotic manipulator.

In accordance with this preferred implementation option, the at least one tendon 31, 32, 33, 34, 35, 36 is mounted to the surgical instrument 20 so as to be operatively connected to both a respective transmission element, among the aforesaid at least one transmission element, and to at least one degree of freedom of the aforesaid at least one degree of freedom P, Y, G of the articulated end-effector 40.

The at least one transmission element is preferably a rigid element. Thereby the action of a motorized actuator is transmitted to the respective tendon without attenuations/distortions which could be instead introduced if the transmission element was an elastic and/or damping element, for example.

The aforesaid at least one degree of freedom P, Y, G operatively connected to the at least one tendon is mechanically activatable by a movement of the aforesaid at least one respective transmission element 21, 22, 23, 24, 25, 26, by the respective at least one tendon 31, 32, 33, 34, 35, 36 operatively connected thereto.

In this embodiment, the method comprises the following steps:
(i) locking at least one degree of freedom of the aforesaid at least one degree of freedom P, Y, G of the articulated end-effector 40;
(ii) tensile-stressing the respective at least one tendon, operatively connected to the at least one locked degree of freedom, by applying a conditioning force Fref, according to at least one time cycle, to the respective transmission element 21, 22, 23, 24, 25, 26 connected to the aforesaid respective at least one tendon to be stressed under tensile load.

The aforesaid at least one time cycle comprises at least one low-load period, in which a low conditioning force Flow is applied to the aforesaid respective transmission element, which results in a respective low tensile load on the respective tendon; and at least one high-load period, in which a high conditioning force Fhigh is applied to the aforesaid respective transmission element, which results in a respective high tensile load on the respective tendon.

Preferably, therefore, the low conditioning force Flow is a force greater than zero and greater than the static sliding friction of the tendon on the surfaces of the surgical instrument, as well as the internal frictions of the actuation and transmission system of the surgical instrument, so as to determine in any case a tensile stress, albeit slight, on the tendon.

In an embodiment, the low conditioning force Flow is in a range of 0.2-1 N. In an embodiment, the low conditioning force Flow is in a range of 1-5 N. In an embodiment, the low conditioning force Flow is in a range of 5-10 N. In an embodiment, the low conditioning force Flow can be a value in a range of 5% to 20% of the high conditioning force Fhigh. In an embodiment, the low conditioning force Flow can be a value in a range of 1% to 10% of the tendon breaking load at the termination thereof (i.e., at the tendon-link constraint point of the surgical instrument). In an embodiment, the low conditioning force Flow can be a value up to 5% of the tendon breaking load.

According to an implementation option, the operating connection between the at least one tendon of the surgical instrument and the respective at least one motorized actuator can be a direct or indirect connection, for example by interposing at least one respective transmission element.

According to an implementation option, the direct or indirect operating connection between the at least one tendon of the surgical instrument and the respective at least one motorized actuator can be a releasable link. For example, the operating connection between the at least one tendon of the surgical instrument and the respective at least one motorized actuator is determined by a detachable monolateral constraint coupling.

According to an embodiment, the method comprises the further steps of:
(iii) unlocking the aforesaid at least one degree of freedom, so that the articulated end-effector 40 can move in all the degrees of freedom thereof;
(iv) performing the teleoperation using the surgical instrument 20. In this case, the conditioning method is understood as a conditioning procedure which is part of a control method which provides the step of carrying out a master-slave teleoperation.

By virtue of the application of the aforesaid force to the at least one transmission element 21, 22, 23, 24, 25, 26, in a condition in which the aforesaid at least one degree of freedom P, Y, G is locked, it allows exerting a tensile action on the aforesaid at least one tendon 31, 32, avoiding however to actuate or activate the aforesaid locked at least one degree of freedom. Thereby, the lengthening/elongation conditions of the at least one tendon 31, 32, 33, 34, 35, 36 are stabilized in view of the next teleoperating step.

Before starting the teleoperating step, the aforesaid at least one degree of freedom of the articulated end-effector 40 is unlocked, in order to become actuatable. For example, “actuatable” means that the degree of freedom is allowed to move in two opposite directions of movement.

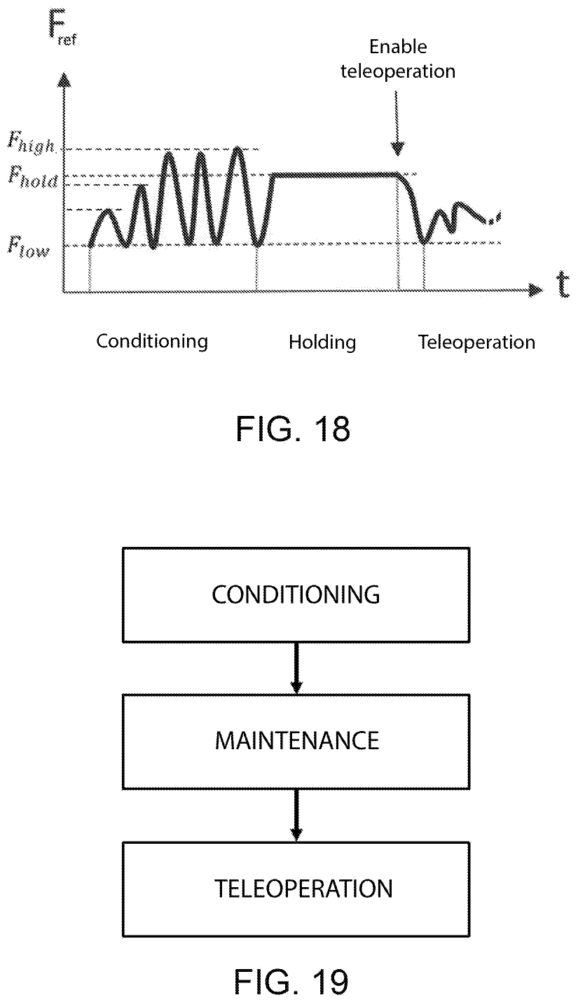

According to an embodiment (shown for example in FIGS. 18 and 19), the method comprises, before step (iv) of performing the teleoperation, the steps of tensile-stressing at least one pair of antagonistic tendons 31, 32; 33, 34; 35, 36 and holding said tendons in a tensile-stressed state by applying a holding force Fhold on the tendons which is adapted to determine a load state on the tendons. These stressing and holding steps form a holding procedure, which follows the conditioning procedure and precedes the teleoperation.

The holding force Fhold is preferably between said low load value Flow and said high load value Fhigh. These tendon stressing and holding steps in a tensile-stressed state can be provided after the unlocking step. Before the stressing step, an unloading or relaxation sub-step can be provided, which can comprise applying a low force on the antagonistic tendons, less than the holding force Fhold and less than the high load Fhigh.

The step of performing a teleoperation can be subject to a request for entry into teleoperation and/or by a command enabling the teleoperation. In other words, after the conditioning procedure a holding step (i.e., a maintenance step) is performed and then a step of performing a teleoperation after the holding step.

Performing the stressing and holding steps allows the tendons of the surgical instrument to be kept taut upon entry into a teleoperating step, ensuring a ready response of the tendons. For example, at the end of the conditioning procedure, the execution of the conditioning procedure allows avoiding the relaxation of the tendons in view of the step of performing a teleoperation, maintaining the level of mechanical conditioning of the tendons reached during the conditioning procedure.

The holding step preferably ends with a relaxation or unloading sub-step, i.e., the application of relatively low tensile force (even null) so as to avoid crushing the tendons before entering the step of performing a teleoperation.

As mentioned above, the conditioning procedure preferably ends with a relaxation or unloading sub-step, i.e., with the application of relatively low (even null) tensile force to the tendons.

Preferably, during the step of performing a teleoperation the surgical instrument follows the master device, for example in all the degrees of freedom of master-slave teleoperation thereof, while during the conditioning and holding procedures the surgical instrument is not enslaved to (i.e., not following) the master device.

In accordance with an implementation option of the method, the locking step comprises bringing to the stroke end (i.e., end-of-stroke) the at least one degree of freedom (P, Y, G) to be locked; and the unlocking step comprises bringing the at least one degree of freedom P, Y, G, to be unlocked in a non-stroke end (i.e., non-end-of-stroke) position.

In accordance with another implementation option of the method, the locking step comprises fitting a constraining element 37 abutting on the articulated end-effector 40; the constraining element 37 is configured to lock one or more of the aforesaid at least one degree of freedom of the articulated end-effector 40 in a precise and predetermined configuration/pose.

In such a case, the unlocking step comprises releasing the articulated end-effector 40 from the condition in which it abuts on the constraining element 37.

In accordance with an embodiment, the method is applicable to a surgical instrument 20 comprising at least one pair of antagonistic tendons (31, 32; 33, 34; 35, 36) comprising the aforesaid at least one tendon, and preferably at least one pair of antagonistic transmission elements (21, 22; 23, 24; 25, 26), among the aforesaid transmission elements.

The at least one pair of antagonistic tendons acts on a single degree of freedom (P; Y; G) associated therewith (i.e., acts on the same rotational joint or on the same link of the end-effector), thus determining antagonistic effects.

Each element of said pair of antagonistic transmission elements, where present, is associated with a respective tendon of said pair of antagonistic tendons.

In such an embodiment, the step of locking a degree of freedom comprises simultaneously activating both antagonistic tendons of the pair of antagonistic tendons associated with the degree of freedom to be locked, so that the respective motors/actuators pull the respective tendon of the antagonistic tendons at the same speed; and the step of unlocking the locked degree of freedom comprises deactivating at least one of the antagonistic tendons of the pair of antagonistic tendons associated with the degree of freedom to be unlocked.

The unlocking step can comprise deactivating both antagonistic tendons associated with the degree of freedom to be unlocked.

According to a preferred implementation option, the locking step of a degree of freedom comprises simultaneously activating both antagonistic transmission elements (21, 22; 23, 24; 25, 26) connected to the pair of antagonistic tendons associated with the degree of freedom to be locked; and the unlocking step of the locked degree of freedom comprises deactivating at least one of the antagonistic transmission elements (21, 22; 23, 24; 25, 26) connected to the pair of antagonistic tendons associated with the degree of freedom to be unlocked.

According to an implementation option, the unlocking step of the locked degree of freedom comprises deactivating both antagonistic transmission elements (21, 22; 23, 24; 25, 26) connected to the pair of antagonistic tendons associated with the degree of freedom to be unlocked.

According to an implementation option, the method provides a plurality of time cycles, in which, in at least two adjacent time cycles, the respective value of the high conditioning force Fhigh grows.

According to an implementation option, the method provides a plurality of time cycles, in which, in at least two adjacent time cycles, the respective value of the high conditioning force Fhigh remains constant.

In accordance with an embodiment of the method, each of the aforesaid at least one tendon 31, 32, 33, 34, 35, 36 is non-elastically deformable under tensile load.

According to various possible implementation options, the aforesaid at least one tendon comprises a plurality of wound/braided polymeric fibers, i.e., it is a polymer strand.

With reference now to the aforesaid time cycles of force applications, if the surgical instrument 20 comprises a plurality of tendons, the method, according to an embodiment, provides that a respective stress pattern (or “diagram”) is applied to each tendon, characterized by respective applied reference forces Fref, and by a respective number of cycles, with respective durations and sequences of the application periods of the high and low conditioning forces.

According to an implementation option, the stress pattern of at least one tendon of the plurality of tendons is different from the stress pattern of the other tendons.

According to another implementation option, the application of the aforesaid conditioning force Fref does not occur simultaneously on all the tendons.

According to a further implementation option, the values of the aforesaid conditioning forces Fref are different on at least one tendon or on different tendons or on different pairs of antagonistic tendons. This applies for example in cases in which the surgical instrument has tendons with different features, for example more robust closing tendons than the others and which can be stressed by higher forces.

In accordance with another implementation option, the aforesaid stress pattern is the same for all the tendons.

In accordance with an implementation option of the method, the application of the aforesaid conditioning force Fref occurs simultaneously on all the tendons.

According to an implementation option, the values of the conditioning forces Fref are the same on all the tendons.

It is apparent from what is described above that the method comprises numerous possibilities for managing the time cycles (in terms of number and duration) and the values and moments of application of forces to the tendons, in a uniform manner on all the tendons, or in a different and individual manner for each tendon, or for any subset of tendons.

Some implementation options of such stress patterns and time cycles are shown in the FIGS. 14-17.

In accordance with a possible operating mode, as diagrammatically shown for example in FIG. 14, the high conditioning force Fhigh is not applied simultaneously on all the tendons, for example the high conditioning force Fhigh is applied first, at a time Ta, on an antagonistic tendon 34 of a pair by means of the respective transmission element 24 and then, at a time Tb, on the other antagonistic tendon 35 of the same pair by means of the respective transmission element 24.

As a result, the antagonistic tendons of a pair can be applied with the same, but also different, and temporally step-dependent stress patterns.

It is also possible to apply the high conditioning force Fhigh simultaneously on the tendons of each pair of antagonistic tendons, but at different times for each particular pair.

In accordance with a possible operating mode, the stress patterns are applied to the tendons at all different times, for example it is applied successively to the tendons; in such a case, the conditioning procedure is performed one tendon at a time.

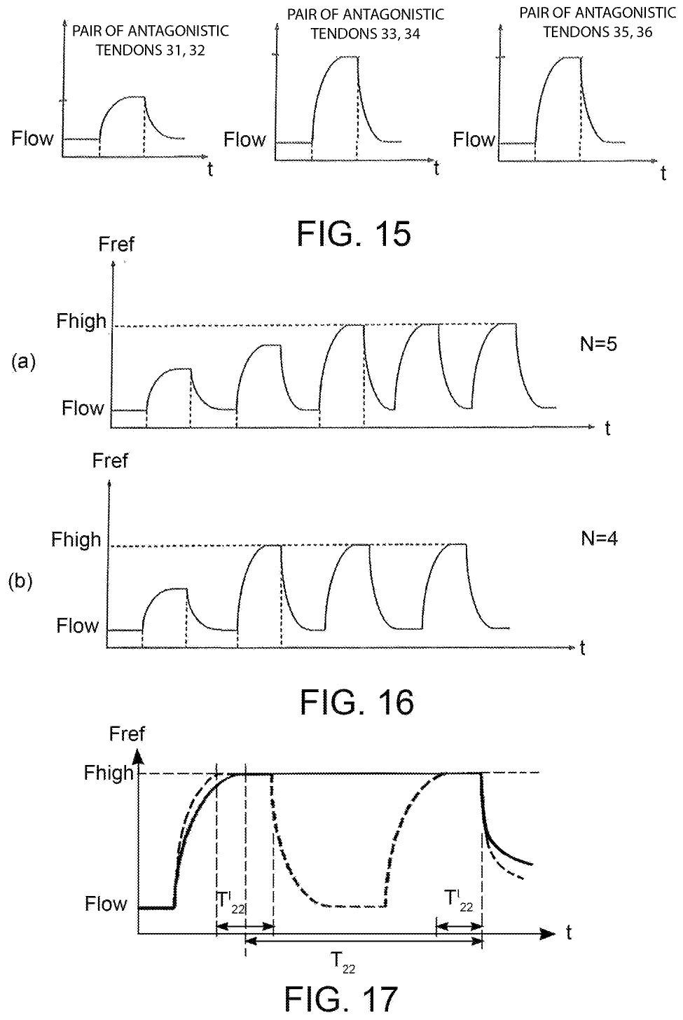

In accordance with a possible operating mode, as diagrammatically shown for example in FIG. 15, the values of the high conditioning force Fhigh are different on at least one tendon or on different tendons or on different pairs of antagonistic tendons, for example the tendons 33, 34, 35, 36 which are involved in the actuation of the degree of freedom of opening/closure G (or “grip” G) can be conditioned with high Fhigh and or low conditioning force Flow values with respect to the tendons 31, 32, and/or with a greater excursion between high conditioning force and low conditioning force Fhigh-Flow.

In accordance with a possible operating mode, a different high conditioning force Fhigh and/or low conditioning force Flow value is applied to the tendons of a pair of antagonistic tendons.

The value of the high conditioning force Fhigh and/or low conditioning force Flow can be chosen based on the mechanical and/or geometric properties as well as the material of the tendon to be stressed. For example, tendons with a larger diameter can be stressed with greater conditioning force.

In accordance with a possible operating mode, the application pattern of the conditioning force can vary between one tendon and another or between a pair of antagonistic tendons and another.

In accordance with a possible operating mode, as shown for example in FIG. 16, a pair of antagonistic tendons (graph a) can be stressed with a greater number of cycles N with respect to another pair of antagonistic tendons (graph b).

In accordance with a possible operating mode, as shown for example in FIG. 17, a pair of antagonistic tendons (continuous lines in the figure) can be stressed with relatively long durations T22 of high conditioning force Fhigh at a force value which remains substantially constant for a duration T22 during which another pair of antagonistic tendons (dashed lines in the figure) has been stressed with two load cycles having duration T′22 less than T22. In accordance with an operating mode, the ascent times T21 and descent times T12 can vary between one tendon and another or between a pair of tendons and another.

As already noted above, in some preferred implementation options, the values of said low conditioning force Flow and high conditioning force Fhigh depend on the physical features of the respective tendon and/or structural features of the instrument related to attachment modes of the tendon with the respective transmission element and/or with the end-effector 40.

This applies for example if the tendons are polymeric, and thus tend to deform (in particular, shorten) over time, and it is thus appropriate and advantageous to operate a preconditioning (as envisaged in the method of the present invention) just before use, so as not to give the tendons time to deform.

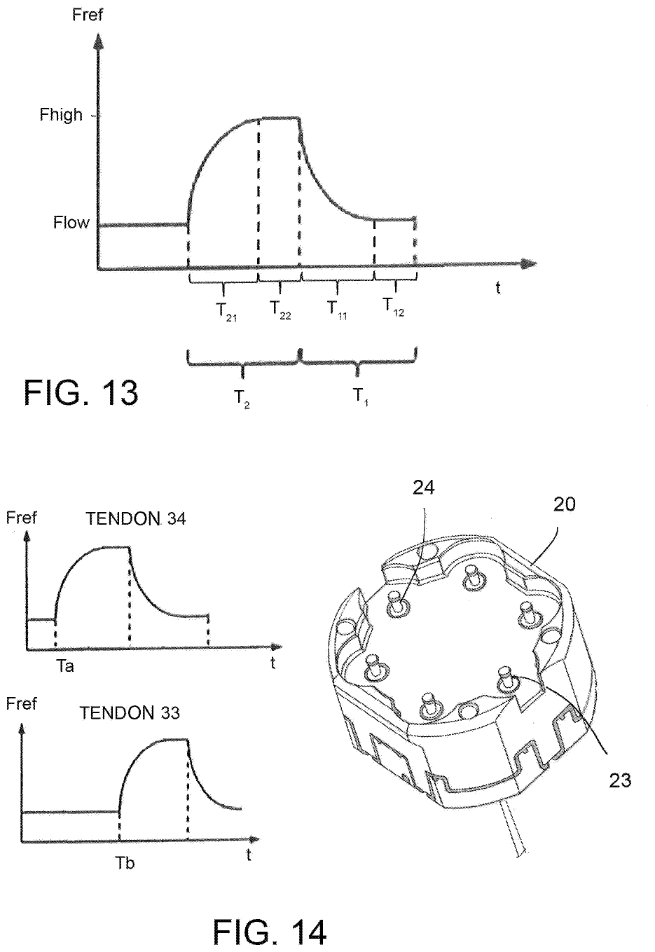

With reference again to the time cycles, according to an embodiment of the method (shown for example in FIG. 13), each of the at least one low-load period has a first time duration T1, and comprises a first holding sub-step having a first holding time duration T12 during which a first force value corresponding to the low conditioning force Flow is applied.

Similarly, each of the at least one high-load period has a second time duration T2, and comprises a second holding sub-step having a second holding time duration T22 during which a second force value corresponding to said high conditioning force Fhigh is applied. According to an embodiment of the method, a plurality of N time cycles is provided, so as to determine an alternation between successive low-load periods and high-load periods, in which during the low-load periods of the n-th cycle a respective low conditioning force Flow_n is applied and in which during the high-load periods of the n-th cycle a respective high conditioning force Fhigh_n is applied.

According to an implementation option, the aforesaid low conditioning forces Flow_n of the different time cycles correspond to a same predetermined value of low conditioning force Flow, and the aforesaid high conditioning forces Fhigh_n correspond to gradually increasing high conditioning force values Fhigh, until reaching a maximum high force value Fhigh_max.

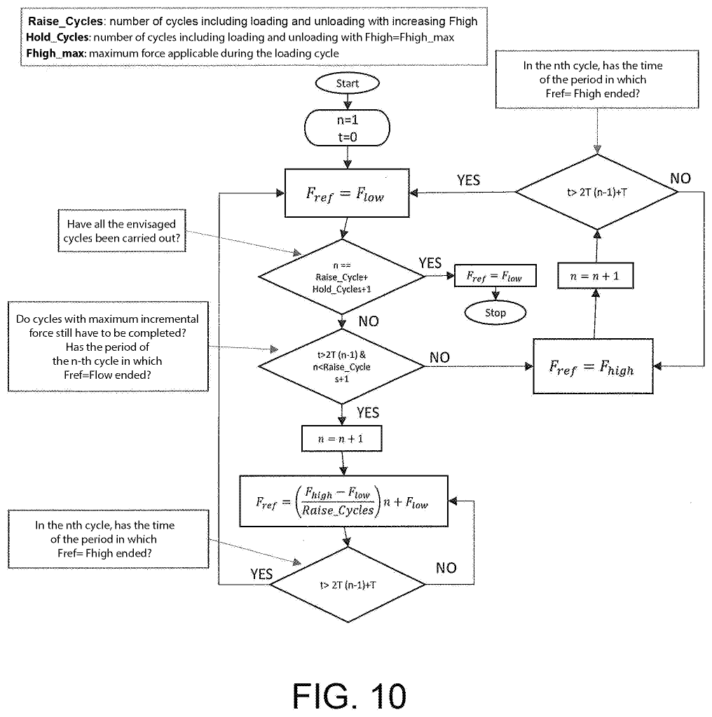

In accordance with an implementation option, the high conditioning force value of the n-th time cycle is calculated according to the following formula:

$${F}_{high \_ n} = \{ \begin{matrix}( \frac{{F}_{high \_ max} - {F}_{low}}{N} ) n + {F}_{low} & n \leq N \\ {F}_{high \_ max} & N < n \leq {N}_{c}\end{matrix} \}$$

where n is the current cycle, N is the total number of cycles in which the force Fhigh is increasing (N is expressed with the variable “Raise Cycles” in FIG. 10), Nc is the number of cycles at constant Fhigh (Nc is expressed with the variable “Hold Cycles” in FIG. 10), and Fhigh_max is a settable value.

In such a case, the high conditioning force values Fhigh are first increasing in constant increments and then constant, according to the above formula.

The flow diagram of FIG. 10 shows in detail an example of how, at the n-th cycle, the forces to be applied, and thus the stress patterns, are calculated.

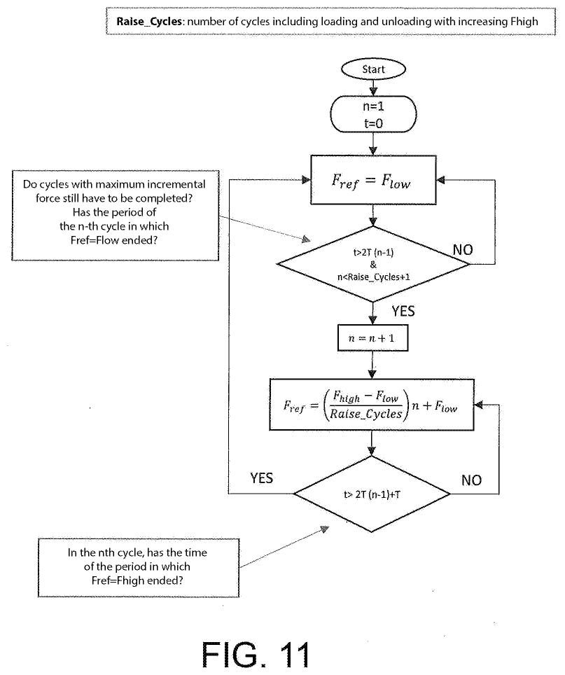

According to another possible operating mode, as shown for example by the diagram of FIG. 11, the high conditioning force values Fhigh are always increasing in constant increments for each cycle, according to the formula:

$${F}_{high \_ n} = ( \frac{{F}_{high \_ max} - {F}_{low}}{N} ) n + {F}_{low}$$

where n is the current cycle, N is the total number of cycles, Fhigh_max is a settable value.

The flow diagram of FIG. 11 shows in detail another example of how, at the n-th cycle, the forces to be applied, and thus the stress patterns, are calculated.

According to other implementation options, the increments of the high conditioning force value Fhigh can be non-constant at each cycle.

According to a particular implementation option, all the time cycles are equal to each other, in which the high conditioning force Fhigh_n of each time cycle corresponds to the aforesaid maximum high force value (Fhigh_max).

According to another implementation option, the method comprises at least one time cycle in which said first time duration T1 is at least five times the aforesaid second time duration T2.

According to another implementation option, the method comprises only one time cycle in which said first time duration T1 is at least five times the aforesaid second time duration T2.

In accordance with an embodiment of the method, said first time duration T1 comprises, in addition to the first holding sub-step with first holding time duration T12, a first ramp sub-step having a first ramp time duration T11, such that the sum of the first holding time duration T12 and the first ramp time duration T11 corresponds to the first time duration T1.

Similarly, said second time duration T2 comprises, in addition to the second holding sub-step with second holding time duration T22, a second ramp sub-step having a second ramp time duration T21, such that the sum of the second holding time duration T22 and the second ramp time duration T21 corresponds to the second time duration T2.

According to an implementation option, the aforesaid first holding time duration T12 is greater than the first ramp time duration T11 and the aforesaid second holding time duration T22 is greater than the second ramp time duration T21. In other words, the holding time is longer than the ascent and descent times.

In accordance with an implementation option of the method, said first time duration T1 is in the range of 0.2 seconds to 30.0 seconds, said second time duration T2 is in the range of 0.2 seconds to 5.0 seconds.

According to an implementation option, preferably, the first time duration T1 is in the range of 1.0 seconds to 3.0 seconds, and the second time duration T2 is in the range of 1.0 seconds to 3.0 seconds.

According to an implementation option, the first ramp time duration T11 is in the range of 0.2 to 10.0 seconds, and the second ramp time duration T21 is in the range of 0.2 to 2.0 seconds.

According to an implementation option, the first holding time T12 is in the range of 0.2 to 20.0 seconds and the second holding time T22 is in the range of 0.2 to 3.0 seconds.

In accordance with an embodiment of the method, said low conditioning force Flow has a value in the range of 0.2 N to 3.0 N, and said high conditioning force Fhigh has a value in the range of 8.0 N to 50.0 N.

According to an implementation option, preferably, the low conditioning force Flow has a value in the range of 1.0 N to 3.0 N, and the high conditioning force Fhigh has a value in the range of 10.0 N to 20.0 N.

In accordance with an embodiment of the method, the aforesaid number N of time cycles is in the range of 1 to 30.

According to an implementation option, preferably, the aforesaid number N of time cycles is in the range between 1 and 15 or is less than 10.

More preferably, according to a particular implementation, the number N of time cycles is in the range of 3 to 8.

In accordance with an embodiment, the low conditioning force Flow changes in the different cycles, and preferably rises with the rise of the high conditioning force Fhigh.

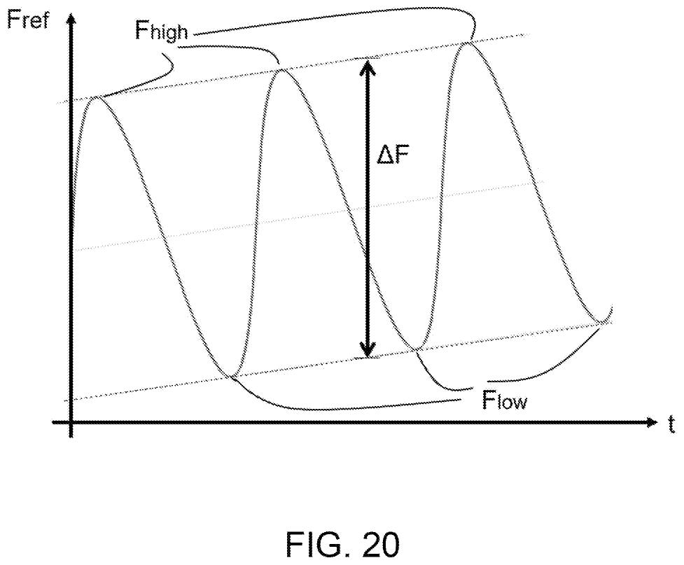

As shown for example in FIG. 20, in accordance with an embodiment of the method, the difference ΔF between the low conditioning force Flow and the high conditioning force Fhigh remains constant during loading and unloading cycles in which the high conditioning force Fhigh grows. In other words, both the high conditioning force Fhigh and the low conditioning force Flow are increasing. Thereby, raising the low force Flow applied to the tendon during loading and unloading cycles minimizes the risk of the tendon undergoing tears and thus possible breakage. Furthermore, thereby, the amplitude of the actuator control (in position or in force, for example) necessary to reach the high force level Fhigh which is increasing in successive cycles, decreases with respect to the case in which the low conditioning force remains constant.

In an implementation option, the low conditioning force Flow grows proportionally as the high conditioning force Fhigh grows. For example, the low conditioning force Flow grows less quickly than the high conditioning force Fhigh. Alternatively, the low conditioning force Flow grows faster than the high conditioning force Fhigh.

Growing the minimum recovery force during subsequent cycles reduces the low-high force range to which the tendon is subjected, and thus reduces the magnitude of the force pulses which could induce possible breakages and in particular if performed at high speed i.e., at high frequency. The amplitude of the position command needed to reach the same force level in subsequent cycles can decrease as well as the process execution time.

With reference to further embodiments of the method, aspects related to controlling the application of conditioning forces to tendons will be described below.

In accordance with an embodiment of the method, the application of the aforesaid low conditioning force Flow, i.e., the low-load period, is associated with a first stroke/position value p1 of the transmission elements; and the application of the aforesaid high conditioning force Fhigh, i.e., the high-load period, is associated with a second stroke/position value p2 of the transmission elements.

In such a case, the method comprises the step of controlling the motorized actuators 11, 12, 13, 14, 15, 16, and if included the transmission elements 21, 22, 23, 24, 25, 26 associated thereto, respectively, in accordance with the respective stress patterns, so that the motorized actuators and/or the transmission elements are in positions corresponding to the first stroke/position value p1 or the second stroke/position value p2, in accordance with the provisions of the respective stress patterns.

According to an implementation option, said respective stress patterns are controlled by means of a feedback loop position control, based on said first and second stroke/position values (p1, p2).

In such a case, the motorized actuators comprise motors operatively connectable to respective transmission elements to impart movement under the control of control means 9 included in the robotic system.

The step of applying the conditioning force Fref and/or the low conditioning force Flow and/or the high conditioning force Fhigh comprises applying the force by a feedback loop control, in which preferably the feedback signal contains information on the detected current position of a motorized actuator and/or a transmission element, and/or the feedback signal contains information on the expected or calculated current position of a motorized actuator and/or a transmission element.

According to an implementation option, the aforesaid respective stress patterns are controlled by means of control with an open loop associated with a predefined number of steps (in this case, in the absence of feedback loops, or force or position detections).

As shown, the above-described embodiment thus operates based on a feedback control based on controlling the position of the actuators and/or the transmission elements.

In accordance with another embodiment of the method, the application of the aforesaid conditioning force is based on a feedback control based on the control of the force itself applied by the motorized actuators to the transmission elements, if present, and/or to the tendons.

In such a case, the robotic surgery system comprises force sensors 17, 18 operatively connectable to at least some transmission elements 21, 22, 23, 24, 25, 26, if present, and preferably said force sensors 17, 18 are located on the respective motorized actuators 11, 12, 13, 14, 15, 16; and the robotic system further comprises control means 9 operatively connected to said force sensors 17, 18.

The motorized actuators 11, 12, 13, 14, 15, 16 comprise motors for imparting movement under the control of the control means. If such transmission elements are included, the motorized actuators 11, 12, 13, 14, 15, 16 comprise motors operatively connectable to respective transmission elements to impart movement to the transmission elements 21, 22, 23, 24, 25, 26 under the control of the control means 9.

The step of applying the conditioning force Fref and/or the low conditioning force Flow and/or the high conditioning force Fhigh comprises applying force by means of a feedback loop control, in which preferably the feedback signal contains information on the applied force as actually detected by a respective force sensor 17, 18.

According to an implementation option, the robotic surgery system comprises a sterile barrier (e.g., a plastic sheet) 19 upstream of the one or more transmission elements 21, 22, 23, 24, 25, 26, i.e., between the sensor of the motorized actuator of the robotic manipulator and the respective transmission element of the surgical instrument.

According to an implementation option, said force sensors 17, 18 comprise respective load cells 17, 18, located in some, and preferably all, of the motorized actuators.

In an implementation example of the method, output controls (“checks”) are provided which are based for example on the information of the current position of the motorized actuators and/or on the information detected by the force sensors 17, 18. For example, if the stroke in the actuation direction of one of the motorized actuators exceeds a safety threshold value, the system can recognize a fault condition aimed at avoiding, for example, reaching the actuator stroke end, as well as the respective transmission element thereof, if included, during the application of the aforesaid conditioning force.

According to an implementation option, the conditioning method is preferably performed shortly before a teleoperating step so as to prevent the tendons from relaxing between the conditioning and teleoperation, and after mounting the instrument to the robotic manipulator and having engaged it to the robotic manipulator.

According to an implementation example, the conditioning method is used to characterize or diagnose the surgical instrument, i.e., to allow the robot to detect from the particular instrument information about the state of the instrument and/or the history of the instrument and/or the expected duration of operation and/or the type of instrument.

Referring again to FIGS. 1-20, a robotic surgery system 1 comprising a surgical instrument 20, at least one motorized actuator 11, 12, 13, 14, 15, 16, and a control unit is described below.

The aforesaid surgical instrument 20 comprises an articulated end-effector 40 having at least one degree of freedom P, Y, G, and further comprises at least one tendon 31, 32, 33, 34, 35, 36, operatively connectable with a respective motorized actuator 11, 12, 13, 14, 15, 16.

The at least one tendon is mounted to the surgical instrument 20 so as to be operatively connectable to both the respective motorized actuator and to at least one degree of freedom of the aforesaid at least one degree of freedom P, Y, G of the end-effector 40. Such an at least degree of freedom P, Y, G is adapted to be mechanically activated by an action of the aforesaid at least one respective motorized actuator 11, 12, 13, 14, 15, 16 by means of the at least one tendon (31, 32, 33, 34, 35, 36) operatively connectable thereto.

The control unit of the robotic surgery system is configured to control the execution of the following actions:
(i) locking at least one degree of freedom of the aforesaid at least one degree of freedom P, Y, G of the end-effector 40;
(ii) tensile-stressing the respective at least one tendon, operatively connected to the at least one locked degree of freedom, by applying a conditioning force (Fref), according to at least one time cycle, to the respective at least one tendon to be stressed under tensile load.

The aforesaid at least one time cycle comprises at least one low-load period, in which a low conditioning force Flow is applied to the respective tendon, which results in a respective low tensile load on the respective tendon, and at least one high-load period, in which a high conditioning force Fhigh is applied to the respective tendon, which results in a respective high tensile load on the respective tendon.

According to an embodiment, the surgical instrument 20 further comprises at least one transmission element 21, 22, 23, 24, 25, 26 operatively connected to a respective at least one tendon 31, 32, 33, 34, 35, 36 and operatively connectable to a respective motorized actuator 11, 12, 13, 14, 15, 16.

In an implementation option, the aforesaid at least one transmission element is a rigid element and said at least one tendon is deformable under tensile load.

According to an embodiment, the robotic surgery system 1 further comprises a sterile barrier interposed between a transmission element and the respective motorized actuator.

According to an implementation option of the system, the operating connection between the at least one tendon 31, 32, 33, 34, 35, 36 of the surgical instrument and the respective at least one motorized actuator 11, 12, 13, 14, 15, 16, is determined by a detachable monolateral constraint coupling.

According to an embodiment, the robotic surgery system 1 further comprises a constraining element 37, fitted on the articulated end-effector 40, and configured to lock one or more of the aforesaid at least one degree of freedom of the articulated end-effector 40 in a precise and predetermined configuration/pose.

According to an embodiment of the system, the aforesaid at least one tendon 31, 32, 33, 34, 35, 36 comprises a plurality of wound/braided polymeric fibers, or is a polymeric strand.

According to various possible embodiments of the system 1, the control unit is configured to control the robotic system so as to perform a method for conditioning a surgical instrument according to any of the previously illustrated embodiments of the method.

Referring again to FIGS. 1-20, further illustrations of the surgical instrument to which the method of the present invention is applied will be provided below, useful for an even better understanding of the method itself, as well as further details, by way of non-limiting example, on some embodiments of the method.

As already observed, according to an embodiment of the method, the conditioning procedure alternately applies low (Flow) and high (Fhigh) force values on at least one tendon 31, 32, 33, 34, 35, 36 of a surgical instrument 20 by applying alternately low (Flow) and high (Fhigh) force values on a transmission element 21, 22, 23, 24, 25, 26 of the surgical instrument 20 associated with said at least one tendon 31, 32, 33, 34, 35, 36. As mentioned above, the motorized actuators can act directly on the tendons or indirectly by interposing the transmission elements.

Such alternately low (Flow) and high (Fhigh) force values can be applied by at least one motorized actuator 11, 12, 13, 14,15, 16 on said at least one transmission element 21, 22, 23, 24, 25, 26.

According to an implementation option, the at least one actuator 11, 12, 13, 14, 15, 16 is a linear actuator.

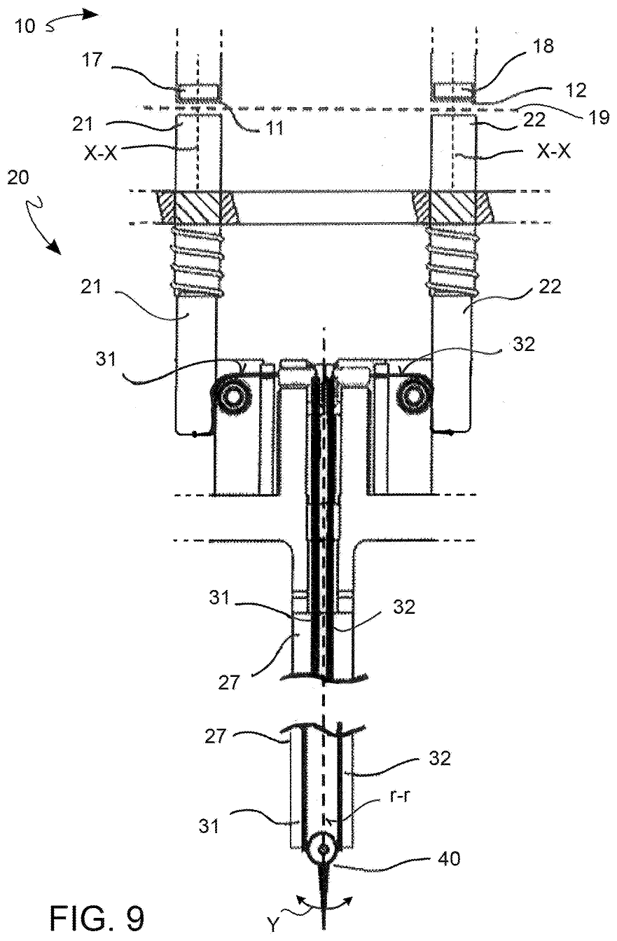

According to an implementation option, the at least one transmission element 21, 22, 23, 24, 25, 26 is a linear transmission element, such as a piston adapted to move along a substantially straight path x-x, as shown for example in FIG. 9.

According to another implementation option, the at least one actuator is a rotary actuator, such as a winch.

According to another implementation option, the at least one transmission element is a rotary transmission element such as a cam and/or a pulley.

Therefore, said alternating low (Flow) and high (Fhigh) force values can be applied when a degree of freedom P, Y, G (P=pitch, Y=yaw, G=grip or opening/closure) actuated by the tendon 31, 32, 33, 34, 35, 36, for example a rotational joint of the surgical instrument 20, is in one of the following conditions:
(i) at the stroke end and/or
(ii) is constrained by a constraining body 37, such as a cap 37 abutting on the articulated end-effector 40.

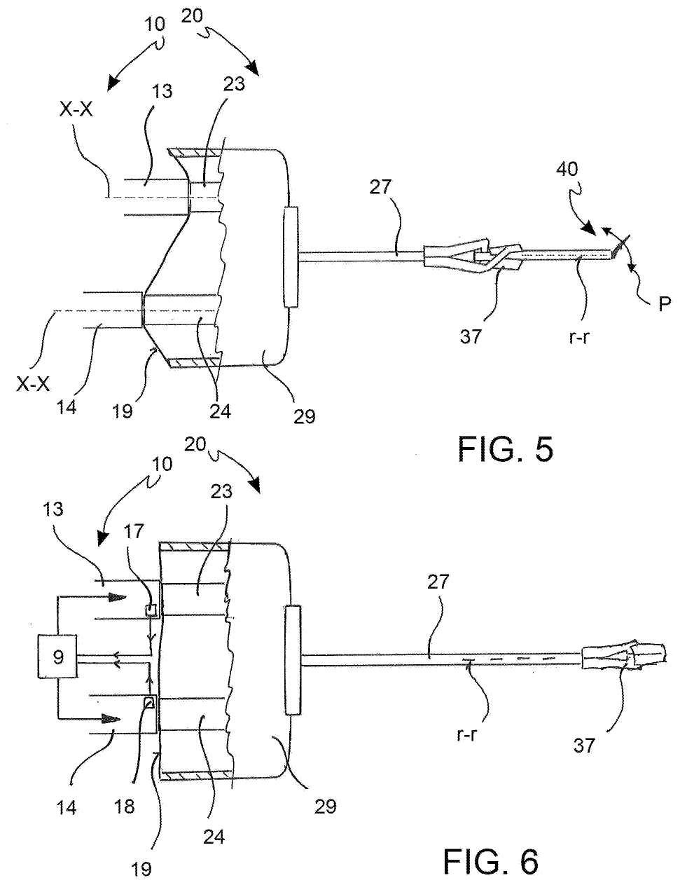

As diagrammatically shown, for example in the sequence of FIGS. 5 and 6, a constraining body 37 (shown here retractable along the shaft 27) can be fitted on the articulated end-effector 40 to lock one or more degrees of freedom (in the example shown, the degree of freedom of pitch P is locked), so as to facilitate the execution of the conditioning procedure.

According to an implementation example, the constraining body 37 is arranged to temporarily lock the articulated tip 40 in a predetermined configuration.

The constraining body 37 can be retractable along the shaft 27 of the surgical instrument 20.

According to various possible options, the constraining body 37 can be a plug 37 or cap 37 which is not retractable along the shaft 27 of the surgical instrument 20, and for example can be removed distally with respect to the free end of the articulated end-effector 40.

The articulated end-effector 40 preferably comprises a plurality of rigid elements (also called “links” 41, 42, 43, 44).

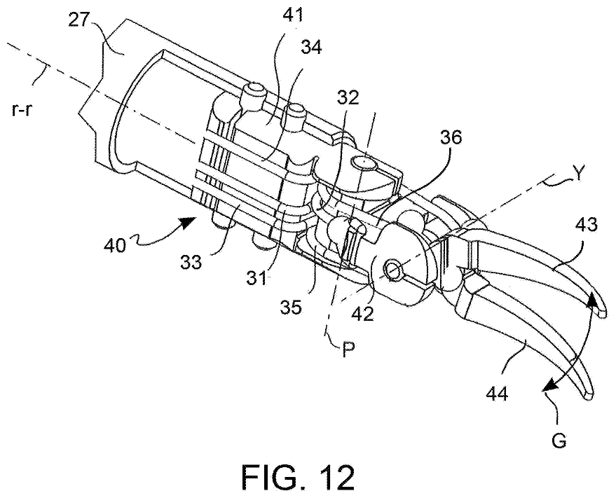

At least some of such links, for example links 42, 43, 44 of FIG. 12, can be connected to a pair of antagonistic tendons 31, 32; 33, 34; 35, 36.

As shown in the embodiment depicted in FIG. 12, a pair of antagonistic tendons 31, 32 can be mechanically connected to a link 42 to move such a link 42 with respect to a link 41 about a pitch axis P, in which the link 41 is shown integral with the shaft 27 of the surgical instrument 20; another pair of antagonistic tendons 33, 34 can be mechanically connected to a link 43 (shown here having a free end) to move said link 43 with respect to the link 42 about a yaw axis Y; another pair of antagonistic tendons 35, 36 can be mechanically connected to a link 44 (shown here having a free end) to move said link 44 with respect to the link 42 about a yaw axis Y; an appropriate joint activation of the links 43 and 44 about the yaw axis Y can determine a degree of freedom of opening/closure or grip G.

Those skilled in the art will appreciate that the configuration of the tendons and links as well as the degrees of freedom of the articulated end-effector 40 can vary from that shown in FIG. 12 while remaining within the scope of the present disclosure.

In accordance with an implementation option, there are three pairs of antagonistic tendons, indicated in the figures with the pairs of numbers (31, 32), (33, 34), (35, 36), configured to actuate three degrees of freedom (for example, the aforementioned degrees of freedom of pitch P, yaw Y and grip G).

In such a case, the surgical instrument 20 can comprise six transmission elements 21, 22, 23, 24, 25, 26 (for example six pistons, as shown for example in FIG. 4), i.e., three pairs of antagonistic transmission elements (21, 22), (23, 24), (25, 26), intended for example to cooperate with three pairs of antagonistic motorized actuators (11, 12) (13, 14), (15, 16). In the example shown in FIG. 4, an exemplary path of the tendons of the three pairs of antagonistic tendons (31, 32), (33, 34), (35, 36) connected to the respective transmission elements (21, 22), (23, 24), (25, 26) is diagrammatically shown in a dashed line, and for example the path of the tendons can be different and extend between reference elements, as diagrammatically shown in FIG. 9, for example.

As already shown above, a sterile barrier 19 can be interposed between the at least one actuator and the at least one transmission element, such as a sterile cloth made as a plastic sheet or other surgically sterile cloth material, such as fabric or non-woven fabric.

According to a preferred embodiment, the conditioning procedure applies the aforesaid alternating low (Flow) and high (Fhigh) force values to at least one pair of antagonistic tendons (31, 32), (33, 34), (35, 36) by applying low (Flow) and high (Fhigh) alternating force values on at least one transmission element associated with said pair of antagonistic tendons (31, 32), (33, 34), (35, 36), and preferably on a pair of antagonistic transmission elements (21, 22), (23, 24), (25, 26) respectively associated with the aforesaid pair of antagonistic tendons (31, 32), (33, 34), (35, 36).

Therefore, the aforesaid alternating low (Flow) and high (Fhigh) force values can be applied when a degree of freedom P, Y, G actuated by the tendon 31, 32, 33, 34, 35, 36 (e.g., a rotational joint of pitch, yaw, and/or grip) of the surgical instrument 20 is under one of the following conditions:
(i) at the stroke end and/or
(ii) is constrained by a constraining body 37 such as a cap 37 abutting on the end-effector 40 of the surgical instrument 20;
(iii) simultaneously actuating the aforesaid pair of antagonistic transmission elements (21, 22), (23, 24), (25, 26), i.e., simultaneously putting both tendons of said pair of antagonistic tendons under tensile load.

According to an embodiment, the conditioning procedure comprises cyclically and alternately applying low Flow and high Fhigh force values on each and on all six transmission elements 21, 22, 23, 24, 25, 26 simultaneously.

It should be noted that the conditioning procedure allows a master-slave teleoperation to be started with a surgical instrument 20 conditioned so as to have the best accuracy as well as optimal performance in terms of grip where the conditioned tendons are those actuating the degree of freedom of grip G. This allows a fine control on the current position of the articulated tip 40 (or end-effector 40) of the slave surgical instrument 20 when in operating conditions.

The force Fref applied by the motorized actuators 11, 12, 13, 14, 15, 16 to the transmission elements 21, 22, 23, 24, 25, 26 results in a tensile action on the respective tendons 31, 32, 33, 34, 35, 36, in which the tensile action on the respective tendons can be substantially equal to the force Fref, although plastic-elastic stretching and/or relaxation behaviors which dissipate a portion of the tensile action can occur.

The at least one tendon is preferably non-elastically deformable, although it can also be elastically deformable.

In accordance with a preferred embodiment, said at least one tendon, and preferably all tendons, of the surgical instrument 20 are made of polymeric material.

Preferably, said at least one tendon, and preferably all tendons, of the surgical instrument 20, comprise a plurality of polymeric fibers wound and/or braided to form a polymeric strand.

In accordance with an embodiment, said at least one tendon comprises a plurality of high molecular weight polyethylene fibers (HMWPE, UHMWPE).

The aforesaid at least one tendon can comprise a plurality of aramid fibers, and/or polyesters, and/or liquid crystal polymers (LCPs), and/or PBOs (Zylon®), and/or nylon, and/or high molecular weight polyethylene, and/or any combination of the foregoing.

According to an implementation option, the aforesaid at least one tendon is made of metallic material, such as a metallic strand.

According to an implementation option, said at least one tendon is made partially of metallic material and partially of polymeric material. For example, said at least one tendon can be formed by the braiding of metallic fibers and polymeric fibers.

In accordance with an embodiment, an electronic controller 9 of the robotic system 1, for example operatively connected to at least one robotic manipulator 10, is configured to monitor the movement of the actuators 11, 12, 13, 14, 15, 16 (e.g., motor pistons) and the conditioning procedure comprises bringing the actuators into contact with the respective transmission elements when the degrees of freedom of the end-effector or articulated tip 40 of the surgical instrument 20 are in a predetermined configuration, for example a configuration in which the links of the articulated tip are aligned along the midline of the instrument and/or the midline r-r of the scope of each degree of freedom. Such a predetermined condition can be when the links of the articulated tip 40 are aligned with the stroke x-x of the transmission elements 21, 22, 23, 24, 25, 26.

It should be noted that the conditioning procedure does not necessarily envisage that the degree of freedom actuated by the at least one tendon is in a predetermined known configuration; such a configuration can be an unknown and/or arbitrary configuration.

According to an one embodiment, the conditioning procedure verifies the following conditions:
condition A) the surgical instrument 20 is correctly engaged on the robotic platform (for example it is housed in a predetermined pocket 28 of the robotic manipulator 10, in which the robotic manipulator comprises the aforesaid motorized actuators 11, 12, 13, 14, 15, 16);
condition B) a plug 37 or cap 37 or constraining body 37 is positioned abutting the end-effector or articulated tip 40 of the surgical instrument 20 locking the joints of the end-effector 40 even if the tendons 31, 32, 33, 34, 35, 36 are taut, i.e., under tensile load;
condition C) the motorized actuators 11, 12, 13, 14, 15, 16 are in contact, for example through a sterile barrier 19, with the tendons and/or with the respective transmission elements 21, 22, 23, 24, 25, 26 if provided.

The contact expressed by such a condition C) can be verified and/or detected by detecting a contact force.

According to an implementation option, already described above, the contact force is detected by one or more load cells 17, 18 (or one or more other force sensors 17, 18) associated with each of the motorized actuators 11, 12, 13,14, 15, 16.

According to other different possible implementation options, condition C) can be obtained by proximity sensors, for example capacitive, or by measurement of the return electromotive force, or by other forms of contact sensing such as BEMF or others.

Condition A) can be verified/or detected by detecting a contact force.

According to an embodiment, a slave robotic system 2 comprises actuators provided with force sensors 17, 18 preferably belonging to at least one robotic manipulator 10.

According to an embodiment, the robotic system is a robotic system for micro-surgical teleoperation, and the surgical instrument is a micro-surgical instrument.

As can be seen, the objects of the present invention as previously indicated are fully achieved by the method described above by virtue of the features disclosed above in detail.

By virtue of the proposed solutions, it is possible to eliminate or at least minimize the recoverable elongation from the at least one tendon to actuate a degree of freedom of the surgical instrument and it is possible to obtain a precise transfer of the actuation action applied on the tendon, independently of the material with which the tendon is formed.

By virtue of the proposed solutions, it is possible to eliminate or at least minimize the permanent lengthening (elongation) which is not recoverable due to the intrinsic structure of the fibers from the at least one tendon to actuate a degree of freedom of the surgical instrument and it is possible to obtain a precise transfer of the action of actuation applied on the tendon, even if the tendon is formed by wound and/or braided polymeric fibers.

By virtue of the proposed solutions, it is possible to stabilize the behavior of the tendons, and thus to stabilize the length of the tendons, before starting a teleoperating step and after mounting the tendon on the surgical instrument of robotic surgery.

By virtue of the proposed solutions, an improved accuracy of the kinematic correspondence between master and slave during a teleoperation.

By virtue of the proposed solutions, lost motion due to an undesirable lengthening/shortening of the tendon when in operating conditions is avoided or at least reduced to a minimum.

By virtue of the proposed solutions, a satisfactory stabilization of the physical features of the surgical instrument is provided.

By virtue of the proposed solutions, improved control over the degrees of freedom of the surgical instrument is provided.

By virtue of the proposed solutions, the necessity of preconditioning the tendons before they are mounted on the surgical instrument is avoided, allowing to perform the conditioning of the tendons just before performing a teleoperating step when the surgical instrument is connected to the robotic platform, further improving the accuracy and precision of the control on the degrees of freedom of the surgical instrument.

By virtue of the provision of conditioning cycles which provide each the application of a low load and the application of a high load, it is possible to make the conditioning of the tendons faster with respect to applying a constant high load.

By virtue of the fact that the high load Fhigh applied during adjacent conditioning cycles grows, this allows not overly stressing the braided tendon, which has a residual plasticity thereof, immediately reaching a maximum load, but instead allows reaching the maximum stress by degrees, thus allowing to remove the plasticity by settling at different increasing load levels and avoiding structural damage or breakage. The above also applies considering the residual plasticity of the multi-component tendon-surgical instrument-motorized actuator product.

Furthermore, increasing steps can reach the same or equivalent plastic recovery levels (deformation) with respect to equal cycles at maximum force, but in reduced time, having to reach smaller levels Fhigh in the first cycles.

By virtue of the fact that the low load Flow applied during adjacent conditioning cycles remains substantially constant and greater than the static friction of the tendon-transmission-actuator system, this allows avoiding any plastic recovery of the tendon. In particular, in fact, the braided polymeric tendons, in particular when they are not under tensile load, due to the braided configuration thereof and the production method by which the fibers are usually crossed under light or high precariousness, have a further plasticity and pure constructional elasticity deriving not from the material or the fiber itself, but from how such fibers are configured (core and jacket), angled (“braiding angle” and “picks-per-inches—ppi”), positioned in section or coupled (double or single), numerous (n fibers) and sized (dtex). Therefore, when they are not under load or tensile load or in particular when they are not subject to even a minimal force Flow, such fibers recover in a characteristic time constant, as a function of the production method and the braiding, the plasticity thereof deriving from the relative rearrangement of the fibers braided together. Such a time constant can also be very fast, whereby in the present invention it is suggested to maintain in the precarious step also a force Flow such as to avoid such plastic recovery.

By virtue of the proposed solutions, it is possible to perform, in preparation for a medical or surgical teleoperation, a procedure for conditioning the polymeric actuation tendons of the surgical instrument which includes the cyclic application of high loads Fhigh which are substantially impulsive or in any case of short duration, followed by a holding procedure in which a holding load Fhold is applied to the actuation tendons for a relatively long duration and greater than the application duration of the high load Fhigh.

To meet contingent needs, those skilled in the art may make changes and adaptations to the embodiments of the method described above or can replace elements with others which are functionally equivalent, without departing from the scope of the following claims. All the features described above as belonging to a possible embodiment can be implemented independently of the other embodiments described.

LIST OF NUMERICAL REFERENCES

| | |
|---|---|
| 1 | Robotic system for teleoperated surgery |
| 2 | Robotic system slave assembly |
| 3 | Master console |
| 9 | Controller, i.e., control unit |
| 10 | Robotic system manipulator |
| 11, 12, 13, 14, 15, 16 | Motorized actuators of the manipulator |
| 17, 18 | Force sensors, or load cells |
| 19 | Sterile barrier |
| 20 | Surgical instrument |
| 21, 22, 23, 24, 25, 26 | Surgical instrument transmission elements |
| 27 | Shaft or rod |
| 28 | Pocket |
| 29 | Surgical instrument backend, or surgical |
| | instrument transmission interface portion |
| 31, 32, 33, 34, 35, 36 | Tendons |
| 37 | Constraining body, or plug, or cap |
| 40 | End-effector device, or articulated tip or |
| | end-effector of the surgical instrument |
| 41, 42, 43, 44 | Links of the articulated end-effector device |
| x-x | Straight direction |
| r-r | Midline |
| P, Y, G | Degree of freedom of the articulated tip |
| | (resp. pitch, yaw, grip) |
| Fref | Applied force |
| Flow | Low force value, or low conditioning force |
| | or low force |
| Fhigh | High force value, or high conditioning force, |
| | or high force |
| Fhold | Holding force value |
| n | Cycle count counter |
| N | Total number of cycles |
| t | Variable “time” |
| T | Total stress pattern time |
| Raise_cycles | Raising cycles |
| Hold_cycles | Holding cycles |

1. A method for conditioning a surgical instrument of a robotic surgery system, wherein the surgical instrument comprises:
an articulated end-effector having at least one degree of freedom;
at least one tendon, which is operatively connectable to a respective at least one motorized actuator of the robotic surgery system,
said at least one tendon being mounted to said surgical instrument to be operatively connectable to both a respective motorized actuator, among said at least one motorized actuator, and at least one degree of freedom among said at least one degree of freedom of the end-effector,
wherein said at least one degree of freedom which is operatively connectable to the at least one tendon is adapted to be mechanically activated by an action of said at least one respective motorized actuator by said at least one tendon which is operatively connectable thereto;
wherein the method comprises:
(i) locking at least one degree of freedom of said at least one degree of freedom of the end-effector;
(ii) tensile-stressing the respective at least one tendon, operatively connected to said at least one locked degree of freedom, by applying a conditioning force, according to at least one time cycle, to the respective at least one tendon to be stressed under tensile load;
wherein said at least one time cycle comprises:
at least one low-load period, in which a low conditioning force is applied to said respective tendon, which results in a respective low tensile load on the respective tendon;
at least one high-load period, in which a high conditioning force is applied to said respective tendon, which results in a respective high tensile load on the respective tendon.

2. A method according to claim 1, wherein the method comprises the further steps of:
tensile-stressing at least one pair of antagonistic tendons;
maintaining said tendons in a tensile-stressed state by applying a holding force to the tendons which is adapted to determine a load state on the tendons.

3. A method according to claim 1, wherein the locking step comprises:
bringing the at least one degree of freedom to be locked to an end-of-stroke; and/or
fitting a constraining element abutting on the articulated end-effector, wherein the constraining element is configured to lock one or more of said at least one degree of freedom of the articulated end-effector in a predetermined configuration/pose,
and wherein the unlocking step comprises:
bringing the at least one degree of freedom to be unlocked to a non-end-of-stroke position; and/or
releasing the articulated end-effector from the condition in which the articulated end-effector abuts said constraining element.

4. A method according to claim 1, wherein the surgical instrument comprises:
at least one pair of antagonistic tendons comprising said at least one tendon, wherein said pair of antagonistic tendons acts on only one degree of freedom associated therewith, thus determining antagonistic effects;
and wherein the robotic surgery system comprises:
at least one pair of antagonistic motorized actuators among said motorized actuators, wherein each element of said pair of antagonistic motorized actuators is associated with a respective tendon of said pair of antagonistic tendons; and wherein:
the step of locking a degree of freedom comprises simultaneously activating both of the antagonistic motorized actuators connected to the pair of antagonistic tendons associated with the degree of freedom to be locked, to pull the respective tendons with a same pulling speed;
the step of unlocking the locked degree of freedom comprises deactivating at least one of the antagonistic motorized actuators connected to the pair of antagonistic tendons associated with the degree of freedom to be unlocked.

5. A method according to claim 1, wherein a plurality of said time cycles is provided, and wherein, in at least two adjacent time cycles, the respective value of the high conditioning force increases.

6. A method according to claim 1, comprising a plurality of said time cycles, and wherein, in at least two adjacent time cycles, a respective value of the high conditioning force remains constant.

7. A method according to claim 1, wherein the surgical instrument comprises a plurality of tendons, and wherein a respective stress pattern is applied to each tendon, said respective stress pattern comprising respective reference forces and respective durations and sequences of the application cycles and/or periods of the high and low conditioning forces,
wherein the stress pattern of at least one tendon of said plurality of tendons is different from the stress pattern of the other tendons,
and/or wherein the application of said conditioning force does not occur simultaneously on all the tendons,
and/or wherein the values of said conditioning forces are different on at least one tendon or different tendons or different pairs of antagonistic tendons,
wherein, the values of the high conditioning force and/or the low conditioning force applied to the tendons which are involved in the actuation of the degree of freedom of opening/closure are higher with respect to the other tendons,
and/or wherein, the excursion between high conditioning force and low conditioning force is greater for the tendons which are involved in the actuation of the degree of freedom of opening/closure.

8. A method according to claim 1, wherein the surgical instrument comprises a plurality of tendons, and wherein a respective stress pattern is applied to each tendon, said respective stress pattern comprising respective reference forces and respective durations and sequences and/or cycles of the application periods of the high and low conditioning forces,
wherein the stress pattern is the same for all the tendons,
and/or wherein the application of said conditioning force occurs simultaneously on all the tendons,
and/or wherein the values of said conditioning forces are the same on all the tendons.

9. A method according to claim 1, wherein a plurality of N time cycles is provided, so as to determine an alternation between successive low-load periods and high-load periods, wherein during the low-load periods of the n-th cycle a respective low conditioning force is applied, and wherein during the high-load periods of the n-th cycle a respective high conditioning force is applied.

10. A method according to claim 9, wherein said low conditioning forces of the different time cycles correspond to a same predetermined low conditioning force value, and wherein said high conditioning forces correspond to gradually increasing high conditioning force values, until reaching a maximum high force value.

11. A method according to claim 10, wherein the high conditioning force value of the n-th time cycle is calculated according to the following formula:
$${F}_{high \_ n} = \{ \begin{matrix}( \frac{{F}_{high \_ max} - {F}_{low}}{N} ) n + {F}_{low} & n \leq N \\ {F}_{high \_ max} & N < n \leq {N}_{c}\end{matrix} \}$$
where n is the current cycle, N is the total number of cycles, Nc is the number of cycles at constant Fhigh, and Fhigh_max is a settable value.

12. A method according to claim 9, wherein:
said first time duration comprises, in addition to the first holding sub-step with first holding time duration, a first ramp sub-step having a first ramp time duration, such that a sum of said first holding time duration and first ramp time duration corresponds to said first time duration;
said second time duration comprises, in addition to the second holding sub-step with second holding time duration, a second ramp sub-step having a second ramp time duration, such that a sum of said second holding time duration and second ramp time duration corresponds to said second time duration,
wherein said first holding time duration is greater than said first ramp time duration and said second holding time duration is greater than said second ramp time duration,
and wherein said first time duration is in the range of 0.2 seconds to 30.0 seconds, and said second time duration is in the range of 0.2 seconds to 5.0 seconds.

13. A method according to claim 12, wherein said first time duration is in the range of 1.0 seconds to 3.0 seconds, and wherein said second time duration is in the range of 1.0 seconds to 3.0 seconds,
and/or wherein said first ramp time duration is in the range of 0.2 to 10.0 seconds and said second ramp time duration is in the range of 0.2 to 2.0 seconds,
and/or wherein said first holding time duration is in the range of 0.2 to 20.0 seconds and said second holding time duration is in the range of 0.2 to 3.0 seconds.

14. A method according to claim 1, wherein said low conditioning force has a positive value greater than a friction value given by a sum of static sliding friction of the tendon on surfaces of the surgical instrument and of internal frictions of actuation and transmission of the surgical instrument, so as to determine in any case a tensile stress on the tendon.

15. A method according to claim 1, wherein:
said low conditioning force has a value in the range of 0.2 N to 3.0 N, and said high conditioning force has a value in the range of 8.0 N to 50.0 N.

16. A method according to claim 1, wherein said number N of time cycles is in the range of 1 to 30.

17. A robotic surgery system comprising a surgical instrument, at least one motorized actuator and a control unit, wherein the surgical instrument comprises:
an articulated end-effector having at least one degree of freedom;
at least one tendon, operatively connectable with a respective motorized actuator of said at least one motorized actuator of the robotic surgery system, said at least one tendon being mounted to said surgical instrument to be operatively connectable to both said respective motorized actuator and to at least one degree of freedom of said at least one degree of freedom of the end-effector,
wherein said at least one degree of freedom which is operatively connectable to the at least one tendon is adapted to be mechanically activated by an action of said at least one respective motorized actuator by said at least one tendon which is operatively connectable thereto;
wherein said control unit of the robotic surgery system is configured to control the performance of the following actions:
(i) locking at least one degree of freedom of said at least one degree of freedom of the end-effector;
(ii) tensile-stressing the respective at least one tendon, operatively connected to said at least one locked degree of freedom, by applying a conditioning force, according to at least one time cycle, to the respective at least one tendon to be stressed under tensile load;
wherein said at least one time cycle comprises:
at least one low-load period, in which a low conditioning force is applied to said respective tendon, which results in a respective low tensile load on the respective tendon;
at least one high-load period, in which a high conditioning force is applied to said respective tendon, which results in a respective high tensile load on the respective tendon.

18. A robotic surgery system according to claim 17, wherein said surgical instrument further comprises at least one transmission element operatively connected to a respective at least one tendon among said tendons and operatively connectable to a respective motorized actuator, wherein said at least one transmission element is a rigid element and said at least one tendon is deformable under tensile load.

19. A robotic surgery system according to claim 18, further comprising a sterile barrier interposed between said at least one transmission element and respective at least one motorized actuator.

20. A robotic surgery system according to claim 17, wherein the operating connection between the at least one tendon of the surgical instrument and the respective at least one motorized actuator is determined by a detachable mono-lateral constraint coupling; or
wherein the robotic surgery system further comprises a constraining element fitted on the articulated end-effector, wherein the constraining element is configured to lock one or more of said at least one degree of freedom of the articulated end-effector in a predetermined configuration/pose, or
wherein said at least one tendon of the surgical instrument comprises a plurality of wound/braided polymeric fibers, or is a polymeric strand.

21-23. (canceled)

24. A method according to claim 1, wherein said low conditioning force has a value in the range of 1.0 N to 3.0 N, and said high conditioning force has a value in the range of 10.0 N to 20.0 N.

25. A method according to claim 1, wherein said number N of time cycles is less than 10.

26. A method according to claim 1, wherein said number N of time cycles is in the range of 3 to 8.
